(19) United States
(12) Patent Application Publication
(10) Pub. No.: US 2026/0309490 A1
(43) Pub. Date: Oct. 8, 2026

(54) DEVICE AND METHOD FOR INSPECTING AT LEAST ONE CONTAINER

(71) Applicant: SIDEL PARTICIPATIONS, Octeville-sur-Mer (FR)

(72) Inventors: Vivien CAVELIER, OCTEVILLE- SUR-MER (FR); Jonas TONGA, OCTEVILLE- SUR-MER (FR)

(21) Appl. No.: 19/637,647

(22) Filed: Apr. 2, 2026

(30) Foreign Application Priority Data
Apr. 3, 2025 (FR) 2503477

(51) Int. Cl. G01B 21/08; G01N 33/00

(52) U.S. Cl. CPC G01B 21/08; G01N 33/0081

(57) ABSTRACT

Disclosed are inspection devices and methods for inspecting containers. The inspection device can include a transport device for transporting the container and at least one inspection module for inspecting the container that comprises at least one acquisition device for acquiring a parameter of the container. The acquisition device is movable in at least three directions: in translation in an acquisition direction, in translation in an elevation direction, and in rotation about an axis of rotation substantially perpendicular to the acquisition direction and to the elevation direction.

### CROSS-REFERENCE TO RELATED APPLICATIONS

This application claims the benefit of French Application No. FR2503477, filed Apr. 3, 2026, the entire contents of which is hereby incorporated herein by reference.

### BACKGROUND

In order to ensure that a container production installation produces containers having the desired characteristics and possibly to remove nonconforming containers, it is known practice to inspect containers at the outlet of the installation by means of an inspection module comprising at least one acquisition device for acquiring at least one parameter of the container, for example the thickness of the wall of a part of the container, and determine if the value of the acquired parameter corresponds to the desired value for this parameter.

In order for the parameter to be correctly acquired, the acquisition device must be placed in a precise position with respect to the container and more particularly with respect to the inspected part of the container. In the case of a wall thickness measurement, the acquisition device must, for example, be placed substantially perpendicularly to the surface of the wall and at a predetermined distance therefrom. Such precise positioning generally poses little problem for containers of circular cross section since the surface of the wall of the container has a substantially uniform shape over the entire body of the container.

However, in the case of a container of more complex shape, for example a container having sharp edges, corners or the like, the positioning of the acquisition device is complicated when it comes to acquiring a parameter relating to a zone of particular shape of the body of the container. Accordingly, it has been found that needs exist for an inspection device making it possible to simply and reliably acquire at least one parameter of the container, whatever the shape of the container. It is to the provision of meeting these and other needs that the present invention is primarily directed.

### SUMMARY

Embodiments of the present disclosure provide an inspection device of the aforementioned type, in which the acquisition device is mounted so as to be movable on the support in at least three directions. The devices is moveable in translation in an acquisition direction in order to distance or move the acquisition device away from the part of the movement path in the acquisition direction, in translation in an elevation direction substantially parallel to the axis of the body of the container when it is moved by the transport device to adjust the height of the acquisition device with respect to the body of the container, and in rotation about an axis of rotation substantially perpendicular to the acquisition direction and to the elevation direction to adjust the orientation of the acquisition device in a plane defined by the acquisition direction and by the elevation direction.

The degrees of freedom in the positioning of the acquisition device on the support allow fine and precise adjustment of the position of the acquisition device with respect to the container transported by the transport device, whatever the shape of the container, in particular in the zone in which the parameter is to be acquired. In addition, when containers of different shape are to be inspected, the new adjustment of the position of the acquisition device can be done quickly and without requiring direct manual action on the inspection module.

An embodiment of the present disclosure includes an inspection device as described above, where the support is additionally rotatable about an axis of rotation substantially parallel to the axis of the body of the container when it is moved by the transport device to adjust the orientation of the acquisition device by modifying the angle formed between the observation direction and the acquisition direction. The acquisition device can be a device for measuring the thickness of a wall of a part of the body of the container.

The inspection module can include at least two acquisition devices mounted on the support, the acquisition devices extending at different heights of the support in the elevation direction, each acquisition device being movable at least in the acquisition direction, in the elevation direction and about an axis of rotation. The inspection device can also include at least two inspection modules each extending opposite a part of the movement path, the inspection modules being spaced apart from one another along the movement path.

The transport device can include a wheel transporting the container along a circular movement path, the inspection module extending opposite an angular sector of the circular movement path. The axis of rotation of the wheel extends in the elevation direction. The inspection module can also include at least a first motor actuating the translational movement of the acquisition device in the acquisition direction, a second motor actuating the translational movement of the acquisition device in the elevation direction, and a third motor actuating the rotational movement of the acquisition device about the axis of rotation.

According to another embodiment, the disclosure also relates to a method for inspecting at least one container by means of an inspection device as described above. The method can include adjusting the position of the acquisition device in the acquisition direction, in the elevation direction and about the axis of rotation such that the acquisition device is in a predetermined position with respect to the body of a container transported along the movement path by the transport device. The method can further include placing the container on the transport device and moving the container along the movement path, the container passing opposite the inspection module during this movement. Next the method can include acquiring at least one parameter of the container by means of the acquisition device when the container extends opposite the acquisition device in the acquisition direction.

According to an optional feature of the inspection method, the acquisition direction extends perpendicularly to the surface of a part of the body of the container in the predetermined position, the parameter of the container acquired by the acquisition device relating to the part of the body of the container.

These and other aspects, objects, features, and embodiments will become apparent to a person of ordinary skill in the art upon consideration of the following detailed description of illustrative embodiments exemplifying the best mode as presently perceived.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 is a schematic top view representation of a device for inspecting containers according to various example embodiments;

FIG. 2 is a schematic side representation of an example of a container that can be inspected by means of the inspection device of FIG. 1;

FIG. 3 is a schematic sectional representation in an elevation direction of an inspection module of the inspection device of FIG. 1; and

FIG. 4 is a functional schematic representation of the inspection module of FIG. 3.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the devices and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20 °C and 1 atmosphere.

Before the embodiments of the present disclosure are described in detail, it is to be understood that, unless otherwise indicated, the present disclosure is not limited to particular materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The features, structures, or characteristics described above may be combined in one or more embodiments in any suitable manner, and the features discussed in the various embodiments may be interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

The terms used herein are intended to have their ordinary meaning unless specifically defined otherwise. Directional terms such as “upper,” “lower,” “front,” “back,” and similar terms are used for convenience and are not intended to be limiting unless the context clearly indicates otherwise. The use of “may,” “can,” “could,” and similar terms indicates possible embodiments and is not intended to limit the scope of the disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “the” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims.

The terms “first,” “second,” etc. are used only as labels, rather than a limitation for a number of the objects. It is understood that if multiple components are shown, the components may be referred to as a “first” component, a “second” component, and so forth, to the extent applicable.

### General Discussion

In accordance with the purpose(s) of the present disclosure, as embodied and broadly described herein, embodiments of the present disclosure, in some aspects, relate to an inspection device for inspecting at least one container comprising a body extending along an axis, the inspection device being of the type comprising a transport device for transporting the container that is designed to move the container along a movement path, and at least one inspection module for inspecting the container that comprises a support extending opposite a part of the movement path in an observation direction substantially perpendicular to the part of the movement path, and at least one acquisition device for acquiring a parameter of the container, the acquisition device being mounted on the support.

The disclosure also relates to a method for inspecting at least one container by means of such an inspection device.

The disclosure applies, for example, to the measurement of the thickness of the wall of a part of a container at the end of a process for producing such a container, for example by stretch blow molding.

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### EXAMPLES

Turning now to the drawings, exemplary embodiments are described in detail.

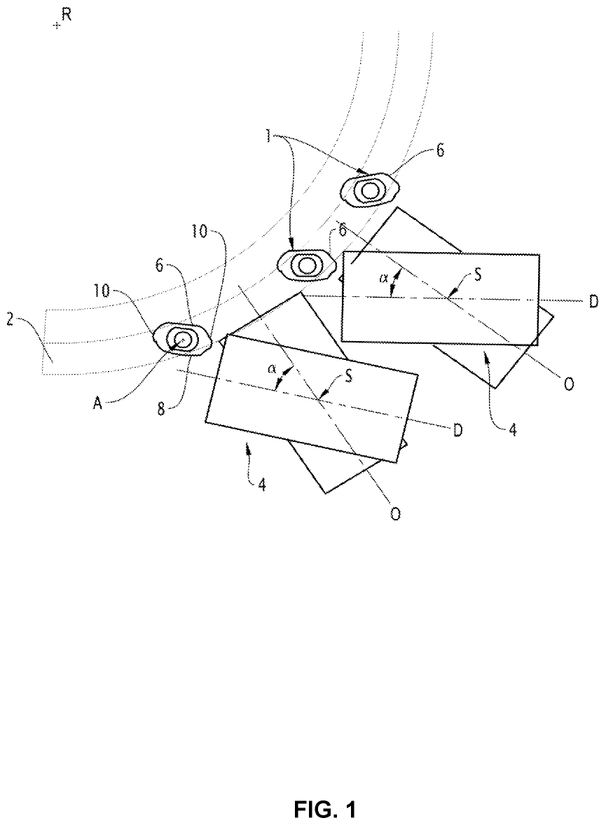

With reference to FIG. 1, there is described an inspection device for inspecting at least one container 1 that comprises a transport device 2 for transporting the container 1 and at least one inspection module 4 for inspecting the container 1. Such an inspection device is, for example, provided in a container production installation in order to verify whether the containers produced conform to a desired container model.

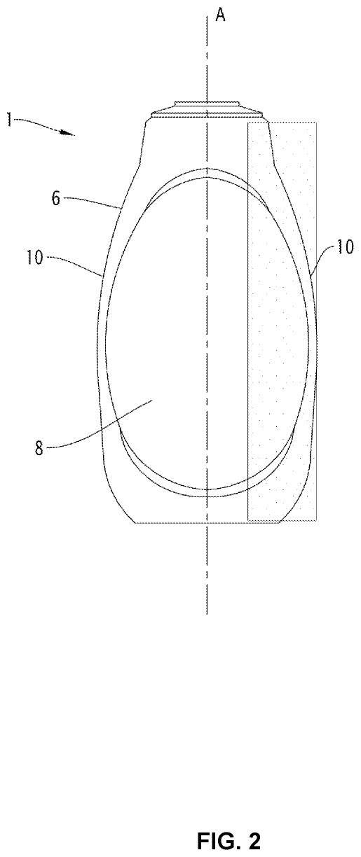

The container 1 comprises a body 6 extending along an axis A, as shown in FIG. 2. The container 1 is, for example, a bottle, a flask or the like. As will be described in greater detail below, the invention is particularly advantageous for inspecting containers having a “complex” shape, in particular the body 6 of which does not have a circular cross section over its entire height measured along the axis A. Thus, as shown in FIGS. 1 and 2, the body 6 of the container 1 has, for example, a cross section of substantially ellipsoidal shape with two substantially planar faces 8 joined together by two lateral portions, for example having a U-shaped, C-shaped or V-shaped cross section and denoted by the term edge faces 10 in the remainder of the description. It is understood that this shape is given only by way of example and that the invention applies to containers of different shapes, including those whose body has a circular cross section.

The transport device 2 is designed to move the container 1 along a movement path. According to one embodiment, the transport device 2 is formed by a wheel rotatable about an axis of rotation R and designed to transport the container 1 at its periphery along a circular movement path. The wheel is designed to maintain the container 1 in a given orientation with respect to its axis A along the movement path, as can be seen in FIG. 1, in which a plurality of containers 1 have been shown. The axis of rotation R of the wheel is then substantially parallel to the axis A of the container 1 when the latter is carried by the wheel. According to one embodiment, the transport device 2 is designed to transport a succession of containers 1 one after another along the movement path. According to one embodiment, the container or containers 1 undergo a particular treatment when they are transported by the transport device 2, before or after being inspected by the inspection module 4. Such a treatment is, for example, the application of a cap or a label to the container 1 or the like.

The inspection module 4 is designed to inspect the container or containers 1 while they are transported along the movement path by the transport device 2. For this purpose, the inspection module 4 is arranged opposite a part of the movement path in an observation direction O. What is to be understood by “opposite a part of the movement path in an observation direction” is that the inspection module 4 is moved away from the movement path in an observation direction O which is substantially perpendicular to the part of the movement path opposite which the inspection module 4 extends. In the case of a circular movement path, the observation direction O is thus substantially perpendicular to the tangent of the part of the movement path opposite which the inspection module 4 extends, as shown in FIG. 1, this part of the movement path being formed by an angular sector of the circular movement path. Thus, the or each container 1 passes in front of the inspection module 4 when it is transported by the transport device 2. More particularly, when the movement path is circular, the or each container 1 passes in front of the inspection module 4 when it circulates in the angular sector forming the part of the movement path opposite which the inspection module 4 extends in the observation direction O.

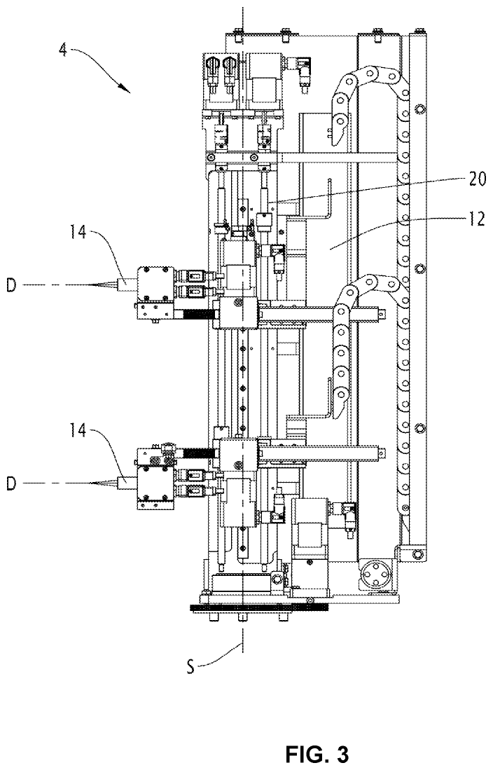

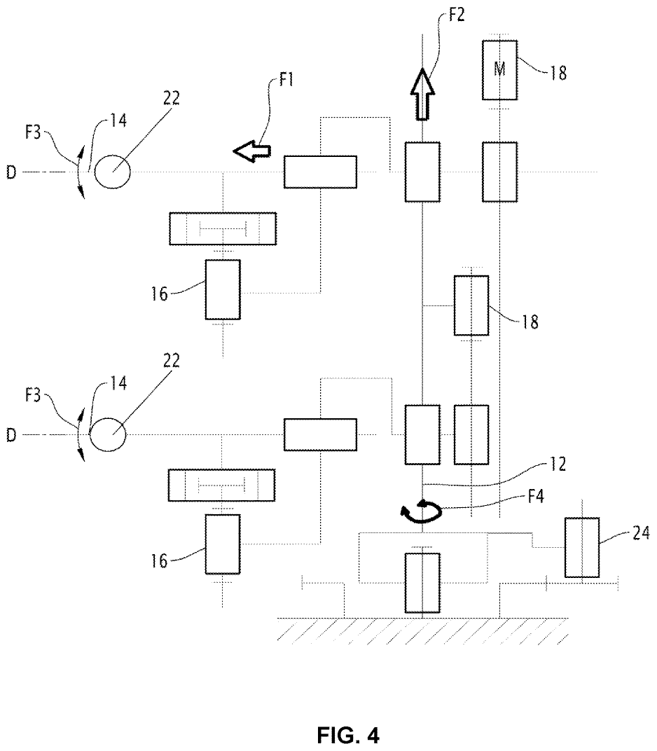

As shown in FIGS. 3 and 4, the inspection module 4 comprises a support 12 and at least one acquisition device 14 mounted on the support 12.

The support 12 forms a frame adapted to receive the acquisition device 14 and also the means allowing the movement of the latter, as will be described below.

The acquisition device 14 is designed to acquire at least one parameter of a container 1 when this container 1 passes in front of the inspection module 4 as described above. The parameter acquired by the acquisition device 14 is, for example, a thickness of a wall of a part of the body 6 of the container 1. Thus, the acquisition device 14 is, for example, a device for measuring the thickness of a wall of a part of the body 6 of the container 1 making it possible to check whether the measured thickness conforms to the thickness desired for this part of the body 6 of the container and thus to check whether the container conforms to the desired container model and/or to a specification. For such a thickness measurement, it is necessary to ensure that the acquisition device 14 acquires the thickness in an acquisition direction D perpendicular to the wall, or to the surface, of the part of the body 6 whose thickness it is desired to acquire. Such a requirement may also apply to other parameters of the container 1.

Thus, the acquisition device 14 must be positioned precisely with respect to the container 1 while the latter is moving along the movement path. This positioning is not particularly complicated when the part of the body 6 of the container 1 for which it is desired to acquire a parameter has a circular cross section since the shape is constant about the axis A, whatever the orientation of the container on the transport device 2. In this case, it is sufficient for the acquisition direction D to be aligned with the observation direction O, since the surface of the body 6 of the container 1 has a tangent parallel to that of the movement path of the container.

On the other hand, as soon as the container 1 has a complex shape, the precise positioning of the acquisition device 14 is more complicated since the shape of the cross section changes around the axis A of the body 6, which therefore changes the acquisition direction D as a function of the part of the body 6 for which it is desired to acquire the parameter. For example, if the part of the body 6 for which it is desired to acquire the parameter is located on the edge face 10 of a container 1 as described above, as shown by the rectangle in dotted lines in FIG. 2, the positioning of the acquisition device 14 is particularly complex because the direction perpendicular to the wall constantly changes along the cross section of the edge face 10. This positioning can be all the more complicated if the edge face 10 has a curved shape, as shown in FIG. 2, the direction perpendicular to the wall also constantly changing along the height of the body 6.

In order to allow a precise placement of the acquisition device 14 with respect to the body 6 of the container 1, the acquisition device 14 is mounted on the support 12 so as to be movable in at least three directions enabling the acquisition device 14 to be positioned in a desired orientation with respect to a part of the body 6 of a container 1 for which it is desired to acquire a parameter, the container 1 being placed on the transport device 2 and passing in the part of the movement path opposite which the inspection module 4 extends.

According to a first degree of freedom, the acquisition device 14 is movable in translation with respect to the support 12 in the acquisition direction D, as shown by the arrow F1 of FIG. 4. In other words, the acquisition device 14 can be moved away from or toward the movement path in the acquisition direction D, which makes it possible to adapt the position of the acquisition device 14 to the radial dimension of the body 6 of the container 1 so that the end of the acquisition device 14 extends at the desired distance from the surface of the part of the body 6 for which it is desired to acquire a parameter. The movement of the acquisition device 14 in the acquisition direction D is, for example, actuated by a first motor 16 mounted on the support 12, as shown schematically in FIG. 4. When the first motor 16 is not actuated, the acquisition device 14 is immobilized in the acquisition direction D. In other words, once the position of the acquisition device 14 has been adjusted in the acquisition direction D, the acquisition device 14 is immobile in the acquisition direction D until a new adjustment is made by means of the first motor 16. Thus, the movement of the acquisition device 14 in the acquisition direction D is not free but constrained to an actuation of the first motor 16.

According to a second degree of freedom, the acquisition device 14 is movable in translation with respect to the support 12 in the elevation direction Z, as shown by the arrow F2 of FIG. 4. In other words, the height of the acquisition device 14 on the support 12 can be adjusted along the axis A of the body 6 of the container 1 when the latter is transported by the transport device 2 as a function of the height of the part of the body 6, measured along the axis A, for which it is desired to acquire a parameter. The movement of the acquisition device 14 in the elevation direction Z is, for example, actuated by a second motor 18 mounted on the support 12, as shown schematically in FIG. 4. When the second motor 18 is not actuated, the acquisition device 14 is immobilized in the elevation direction Z. In other words, once the position of the acquisition device 14 has been adjusted in the elevation direction Z, the acquisition device 14 is immobile in the elevation direction Z until a new adjustment is made by means of the second motor 18. Thus, the movement of the acquisition device 14 in the elevation direction Z is not free but constrained to an actuation of the second motor 18. As shown in FIG. 3, the inspection module 4 comprises, for example, a transmission element 20 between the second motor 18 and the acquisition device 14, enabling the acquisition device 14 to be moved in the elevation direction Z when the second motor 18 is actuated. Such a transmission element 20 is, for example, formed by a ball screw in FIG. 3.

According to a third degree of freedom, the acquisition device 14 is movable in rotation with respect to the support 12 about an axis of rotation substantially perpendicular to the acquisition direction and to the elevation direction, as shown by an arrow F3 of FIG. 4. In other words, the orientation of the acquisition device 14 can be adjusted in a plane containing the acquisition direction D and the elevation direction Z in order to modify the acquisition direction D in this plane. Such an orientation makes it possible to point the acquisition device upward or downward in order to adapt the acquisition direction D to the shape of the part of the body 6 of the container 1 for which it is desired to acquire a parameter. Such an orientation makes it possible in particular to take account of the curvature of the body 6, for example at the edge face 10 as shown in FIG. 2, in order to ensure that the acquisition direction is perpendicular to the desired part of the body 6, including when the latter does not extend in a plane parallel to the axis A of the body 6. The movement of the acquisition device 14 about its axis of rotation is, for example, actuated by a third motor 22 mounted on the support 12, as shown schematically in FIG. 4. When the third motor 22 is not actuated, the acquisition device 14 is immobilized about its axis of rotation. In other words, once the position of the acquisition device 14 has been adjusted about its axis of rotation, the acquisition device 14 is immobile about this axis until a new adjustment is made by means of the third motor 22. Thus, the movement of the acquisition device 14 about its axis of rotation is not free but constrained to an actuation of the third motor 22.

According to one embodiment, when the containers 1 to be inspected are moved by the transport device 2 in such a way that at least one part for which a parameter is desired to be acquired does not extend parallel to the tangent to the circulation path in the angular sector opposite the inspection module 4, the orientation of the acquisition direction D with respect to the observation direction O is also adjustable, as shown in FIG. 1. To this end, according to this embodiment, the support 12 of the inspection module 4 is movable in rotation about an axis of rotation S substantially parallel to the axis A of the body 6 of the container 1 when it is moved by the transport device 2 to adjust the orientation of the acquisition device 14 by modifying the angle α formed between the observation direction O and the acquisition direction D, as shown in FIG. 1 and as shown by the arrow F4 of FIG. 4. In this figure, the position of the inspection module 4 when the acquisition direction D coincides with the observation direction O is shown in dotted lines and the position in which the acquisition direction D forms a nonzero angle α with the observation direction O is shown in solid lines. The movement of the support 12 about its axis of rotation S is, for example, actuated by a fourth motor 24, as shown schematically in FIG. 4. When the fourth motor 24 is not actuated, the support 12 is immobilized about its axis of rotation S. In other words, once the position of the support 12 has been adjusted about its axis of rotation S, the support 12 is immobile about this axis until a new adjustment is made by means of the fourth motor 24. Thus, the movement of the support 12 about its axis of rotation S is not free but constrained to an actuation of the fourth motor 24.

The possible adjustments of the inspection module 4 described above thus make it possible to precisely position the acquisition device 14 so that the parameter acquired by the latter is optimal by orienting the acquisition direction D so that it is strictly perpendicular to the surface of the part of the body 6 of the container 1 for which it is desired to acquire the parameter. These adjustments are made for a container 1, for example placed opposite the inspection module 4 on the transport device 2 without the latter moving the container 1. Once these adjustments have been made by actuating the various motors, the acquisition device 14 is in a predetermined fixed position and the inspection of one or more containers 1 can start. To do this, a succession of containers 1 is placed on the transport device 2 and travels in front of the inspection module 4 with the acquisition device 14 in the predetermined position. The latter then acquires the desired parameter for each container 1. This acquisition is done for the same part of the body 6 of all the containers so that repeatability of the measurement of the desired parameter is ensured while the containers move at high speed along the movement path. Such an inspection of the containers 1 makes it possible to ensure that the containers 1 conform to a desired container model and/or to a specification, and the container or containers whose acquired parameter does not correspond to an expected parameter or is not within an acceptable range of values for this parameter may be discarded.

When the type of containers to be inspected is modified, the position of the acquisition device 14 can be adjusted again rapidly thanks to the various motors of the inspection module 4 and without an operator needing to act manually directly on the inspection module 4 to move and then immobilize the acquisition device 14 on the support 12.

According to the embodiment shown in FIGS. 3 and 4, the inspection module 4 comprises at least two acquisition devices 14 as described above. These acquisition devices 14 are mounted on the support 12 at different heights of the support 12 in the elevation direction Z. Each acquisition device 14 is then movable at least in the acquisition direction D, in the elevation direction Z and about an axis of rotation, as described above, for example by means of dedicated motors for each acquisition device 14. The orientation of the acquisition direction D of each acquisition device 14 is preferably adjustable independently of that of the acquisition direction of the other acquisition device or devices 14 in order to take account of any variations in the shape of the body 6 of the container 1 along its axis A. The acquisition devices 14 of the inspection module 4 may be provided to acquire the same parameter at different locations of the body 6 or different parameters.

According to the embodiment shown in FIG. 1, the inspection device comprises at least two inspection modules 4, each comprising a support 12 on which one or more acquisition devices 14 are mounted. The inspection modules 4 are spaced apart from one another along the movement path of the containers so as to be placed opposite different parts of this movement path. Such an embodiment makes it possible in particular to inspect one and the same region of a container 1, taking into account the fact that this container 1 can rotate about its axis A while it is transported by the transport device 2. To this end, in addition to the possible movements of each acquisition device 14, the support 12 of each inspection module 4 is, for example, movable in rotation about its respective axis of rotation S. Preferably, the adjustment of the position of the support 12 of an inspection module 4 about its axis of rotation S can occur independently of this adjustment for the other inspection module or modules 4. Thus, the inspection modules 4 can be used to acquire one or more parameters for one or more same parts of the body 6 of containers 1 or for different parts of the body 6 of containers 1, taking into account a change in orientation of the containers 1 while they move along the movement path.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

Therefore, at least the following is claimed:

1. An inspection device for inspecting at least one container comprising a container body extending along an axis (A), the inspection device comprising:
a transport device for transporting the container, wherein the transport device is configured to move the container along a movement path;
at least one inspection module for inspecting the container, wherein the inspection module comprises a support extending opposite a part of the movement path in an observation direction (O) substantially perpendicular to the part of the movement path; and
at least one acquisition device for acquiring a parameter of the container, the acquisition device being mounted on the support, wherein the acquisition device is mounted so as to be movable on the support in at least three directions:
in translation in an acquisition direction (D) in order to distance or move the acquisition device away from the part of the movement path in the acquisition direction (D),
in translation in an elevation direction (Z) substantially parallel to the axis (A) of the body of the container when the container is moved by the transport device to adjust a height of the acquisition device with respect to the body of the container, and
in rotation about an axis of rotation substantially perpendicular to the acquisition direction (D) and to the elevation direction (Z) to adjust an orientation of the acquisition device in a plane defined by the acquisition direction (D) and by the elevation direction (Z).

2. The inspection device as claimed in claim 1, wherein the support is additionally rotatable about an axis of rotation(S) substantially parallel to the axis (A) of the container body when the container is moved by the transport device to adjust an orientation of the acquisition device by modifying an angle (α) formed between the observation direction (O) and the acquisition direction (D).

3. The inspection device as claimed in claim 1, wherein the acquisition device is a device configured to measure a thickness of a wall of a part of the body of the container.

4. The inspection device as claimed in claim 1, wherein the inspection module comprises at least two acquisition devices mounted on the support, the acquisition devices extending at different heights of the support in the elevation direction (Z), each acquisition device being movable at least in the acquisition direction (D), in the elevation direction (Z), and about an axis of rotation.

5. The inspection device as claimed in claim 1, comprising at least two inspection modules each extending opposite a part of the movement path, the inspection modules being spaced apart from one another along the movement path.

6. The inspection device as claimed in claim 1, wherein the transport device is a wheel transporting the container along a circular movement path, the inspection module extending opposite an angular sector of the circular movement path.

7. The inspection device as claimed in claim 6, wherein the axis of rotation (R) of the wheel extends in the elevation direction (Z).

8. The inspection device as claimed in claim 1, wherein the inspection module comprises at least a first motor actuating translational movement of the acquisition device in the acquisition direction (D), a second motor actuating the translational movement of the acquisition device in the elevation direction (Z), and a third motor actuating rotational movement of the acquisition device about the axis of rotation.

9. A method for inspecting at least one container by via an inspection device, where:
the inspection device comprises:
a transport device for transporting the container, wherein the transport device is configured to move the container along a movement path;
at least one inspection module for inspecting the container, wherein the inspection module comprises a support extending opposite a part of the movement path in an observation direction (O) substantially perpendicular to the part of the movement path; and
at least one acquisition device for acquiring a parameter of the container, the acquisition device being mounted on the support, wherein the acquisition device is mounted so as to be movable on the support in at least three directions:
in translation in an acquisition direction (D) in order to distance or move the acquisition device away from the part of the movement path in the acquisition direction (D),
in translation in an elevation direction (Z) substantially parallel to the axis (A) of the body of the container when the container is moved by the transport device to adjust a height of the acquisition device with respect to the body of the container, and
in rotation about an axis of rotation substantially perpendicular to the acquisition direction (D) and to the elevation direction (Z) to adjust an orientation of the acquisition device in a plane defined by the acquisition direction (D) and by the elevation direction (Z); and
where the method comprises the following steps:
adjusting a position of the acquisition device in the acquisition direction (D), in the elevation direction (Z), and about the axis of rotation such that the acquisition device is in a predetermined position with respect to the body of a container transported along the movement path by the transport device;
placing the container on the transport device and moving the container along the movement path, the container passing opposite the inspection module during this movement; and
acquiring at least one parameter of the container via the acquisition device when the container extends opposite the acquisition device in the acquisition direction (D).

10. The method as claimed in claim 9, wherein the acquisition direction (D) extends perpendicularly to the surface of a part of the body of the container in the predetermined position, the parameter of the container acquired by the acquisition device relating to the part of the body of the container.
